(19) United States
(12) Patent Application Publication
(10) Pub. No.: US 2024/0218331 A1
(43) Pub. Date: Jul. 4, 2024

(54) METHOD FOR PRODUCING VASCULARIZED BIOLOGICAL TISSUE

(71) Applicants: Fraunhofer-Gesellschaft zur Foerderung der angewandten Forschung e. V., Muenchen (DE); Universitaet des Saarlandes, Saarbruecken (DE)

(72) Inventors: Heiko ZIMMERMANN, Sulzbach (DE); Michael GEPP, Wuerzburg (DE); Benjamin FISCHER, Muenchen (DE)

(21) Appl. No.: 18/558,226

(22) PCT Filed: Apr. 27, 2022
(86) PCT No.: PCT/EP2022/061249

§ 371 Date: Oct. 31, 2023

(30) Foreign Application Priority Data
May 7, 2021 (DE) 10 2021 111 956.1

(51) Int. Cl. C12N 5/077; A61L 27/20; A61L 27/22; A61L 27/38; B33Y 10/00; B33Y 80/00

(52) U.S. Cl. CPC C12N 5/0657; A61L 27/20; A61L 27/22; A61L 27/3808; A61L 27/3826; B33Y 10/00; B33Y 80/00; A61L 2400/08; A61L 2430/20; C12N 2513/00; C12N 2533/50; C12N 2533/74

(57) ABSTRACT

The invention relates to a method for producing vascularized biological tissue, having the steps of producing a network structure made of a plurality of interconnected filaments (11) of a support polymer, coating the network structure with a protein material, populating the coated network structure with endothelial cells (2, 2A) and tissue-forming biological cells (3), and dissolving the filaments (11) such that the vascularized tissue (1) is formed. The vascularized tissue (1) comprises cardiomyocytes, liver cells, renal cells, nerve cells, and/or pancreatic cells, for example.

The invention relates to a method for producing biological tissue, in particular tissue from cardiac muscle cells, liver cells, kidney cells, nerve cells and/or pancreatic cells. Applications of the invention are in biomedicine, for example, in particular in so-called tissue engineering.

It is generally known to replicate biological tissue, comprising a large number of differentiated biological cells having a common function, for research purposes or for implantation purposes using biotechnological methods. For example, cardiac muscle cells can be obtained by differentiation from pluripotent stem cells and propagated by culturing in an incubator. However, conventional replicas of biological tissue (tissue products), which comprise an accumulation of differentiated cells, have the following disadvantages, which result in limitations in the application of tissue products and the costs thereof.

First of all, natural biological tissue contains not only the differentiated cells, but also blood vessels and an extracellular matrix. Only in association with the further tissue constituents does the accumulation of differentiated cells provide an adaptation to the properties of natural biological tissue. Therefore, replications of biological tissue have hitherto been able to perform the functions of natural tissue to only a limited extent. Furthermore, the size of conventional replicas of biological tissue is limited, because cells inside a cell culture may not be able to be supplied optimally with nutrients and oxygen and die. In particular in the case of culturing in the adherent state, conventional replicas of biological tissue frequently have a layer form, which is not optimally adapted to the natural spatial form of a tissue. When conventional tissue products are implanted into a biological organism, undesirable immune responses or even rejection reactions can occur, since the tissue products are recognized as foreign materials because their composition differs from that of the natural tissues.

The objective of the invention is to provide an improved method for producing biological tissue, with which limitations of conventional techniques are avoided. The method for producing biological tissue is to be characterized in particular by the creation of a tissue, the properties of which are better adapted to properties of a natural tissue, which can be produced with larger dimensions, which can be produced with a freely selectable spatial form, which is characterized by reduced immune responses, which can be produced more inexpensively and/or which makes possible new or expanded applications of tissue products.

This objective is achieved by a method for producing biological tissue which has the features of claim 1. Preferred embodiments and applications of the invention will become apparent from the dependent claims.

According to a general aspect of the invention, the above objective is achieved by a method for producing vascularized biological tissue. The method comprises the steps of producing a network structure from a plurality of interconnected filaments of a support polymer, coating the network structure with a protein material, populating the coated network structure with endothelial cells and with tissue-forming biological cells, and dissolving the filaments of the network structure so that the vascularized tissue is formed.

Advantageously, there is provided by means of the coated network structure a single—or multi-layer volume substrate which is formed by the filaments of the support polymer. The filaments, which may also be referred to as threads or fibers or elongate polymer sections, form, for example, a disordered distribution or a regular distribution in space, in which the filaments are in contact with one another or spaced apart from one another at some points or in some sections. There is thus created an inner surface of the network structure which makes it possible for the network structure to be coated with the protein material and subsequently populated with the vessel cells (e.g. endothelial cells) and with the tissue-forming biological cells, optionally with the addition of extracellular matrix (ECM). The filaments are preferably produced from a polysaccharide, such as, for example, alginate or gelatin. Coating of the network structure comprises in particular coating the filaments inside the network structure with the protein material. The protein material advantageously promotes adherent growth of the cells on the filaments.

In order to populate the coated network structure with the endothelial cells and the tissue-forming biological cells, the network structure is incubated with a cell suspension, so that the cells adhere in an adherent manner to the filaments, and the adhered cells are cultured, preferably with a supply of culture medium (nutrient medium). Preferably, the endothelial cells are introduced first into the network structure and then, after a layer (multilayer, monolayer or sub-monolayer) of endothelial cells has formed, the tissue-forming biological cells are introduced.

The endothelial cells are differentiated cells which form the inside of the blood vessels in natural tissue. Preference is given to the use of endothelial cells of the organism, for example a mammal, in particular a human being, whose tissue is to be replicated. The tissue-forming biological cells are differentiated cells of at least one cell type of which the tissue to be replicated is to be composed. Differentiation of the tissue-forming biological cells preferably takes place before the network structure is populated.

It is provided that the network structure is dissolved after the network structure has been populated with the cells. Dissolution of the network structure preferably comprises a chemical and/or thermal conversion of the filaments into a liquid state and flushing of the converted material out of the populated cell composite. By dissolving the network structure, interconnected hollow spaces remain in the composite of cells, the inner surfaces of which hollow spaces are formed by the endothelial cells. The inventors have found that these hollow spaces are distributed geometrically in the same way as blood vessels in natural tissue and accordingly form blood vessels in the vascularized tissue which preferably constitutes the finished tissue product.

The network structure is preferably dissolved after population is complete and the inner volume of the network structure is densely filled with the cells. Alternatively, dissolution can be provided when the inner volume of the network structure is only partly filled, so that the growth of further vessels is facilitated.

The following advantages are advantageously achieved with the vascularized tissue produced according to the invention.

The properties of the vascularized tissue are better adapted to properties of the corresponding natural tissue than is the case with conventional tissue products. The vascularized tissue may even be indistinguishable from natural tissue. A reduction in immune responses on implantation of the tissue product into a living organism is accordingly also possible.

Furthermore, the size limitations of conventional tissue products are overcome in that the differentiated cells in the tissue can be supplied through the vessels in the vascularized tissue. The vascularized tissue is not limited to a layer form but can be produced with a freely selectable spatial form which is adapted to the form of the natural tissue or of a desired implant.

The method according to the invention has a positive impact on the costs of the vascularized tissue, which can be produced in larger volumes and in larger quantities with increased efficiency than conventional tissue products.

The vascularized tissue produced according to the invention also offers novel applications of tissue products in particular in research and in implantation medicine, for example in the creation of model tissues, which do not differ or differ only negligibly from natural tissue.

The invention can be used with various tissue-forming cells. According to a preferred application of the invention, the tissue-forming cells comprise cardiac muscle cells and the vascularized tissue a cardiac muscle tissue product. Alternatively, the tissue-forming cells comprise liver cells, kidney cells, nerve cells or pancreatic cells, the vascularized tissue correspondingly being a liver tissue product, kidney tissue product, nerve tissue product or pancreatic tissue product. The vascularized tissue may alternatively contain multiple cell types, such as, for example, cardiac muscle cells and nerve cells or other cells in combination with nerve cells.

Advantageously, various embodiments of the invention are available in which the network structure is produced on a solid carrier substrate or without binding to a solid carrier substrate.

According to a preferred embodiment of the invention, the production of the network structure comprises depositing the filaments on a carrier substrate coated with a degradable matrix material, such as, for example, a polysaccharide, in particular dextran, and subsequently dissolving the network structure from the carrier substrate. The network structure is advantageously supported by the carrier substrate.

Depositing the filaments comprises arranging a plurality of previously produced, for example extruded, filaments of the support polymer and/or depositing the support polymer by means of a 3D deposition method, in such a manner that the filaments are built up on the carrier substrate. A 3D deposition method includes, for example, a 3D printing method, in particular 3D freeze printing, in which the filaments of the support polymer are formed in the frozen state.

The carrier substrate can be coated with the matrix material in a preparation step immediately before the deposition of the filaments. There is preferably used as the carrier substrate the base of a vessel, which in the subsequent steps of coating the filaments and populating with cells receives solutions or suspensions of the supplied components and forms an incubator. The vessel is, for example, a reaction vessel of a reaction plate, such as, for example, a microtiter plate.

The matrix material forms an isolation layer. Dissolving the network structure from the carrier substrate comprises dissolving the matrix material. The matrix material preferably differs in terms of its chemical solubility from the solubility of the filaments. The filaments and the matrix material are particularly preferably produced from different polysaccharides. Advantageously, the filaments are thus initially retained when the network structure is dissolved from the carrier substrate.

Accordingly, in this embodiment of the invention, preferably a two-stage dissolution of the matrix material and of the support polymer, particularly preferably of polysaccharides, is provided, in which the network structure is first dissolved from the carrier substrate and then, after population with the cells, the network structure is dissolved.

According to a particularly preferred embodiment of the invention, it is provided that a network structure-cell composite is formed by coating the network structure with the protein material and populating the coated network structure with the endothelial cells and with the tissue-forming biological cells, before the network structure is dissolved from the carrier substrate, wherein lateral sections of the filaments in the network structure-cell composite (i.e. a lateral surface of the network structure-cell composite) touch the carrier substrate, wherein subsequently the steps of dissolving the network structure-cell composite from the carrier substrate, folding the network structure-cell composite to form a multilayer, in such a manner that the lateral sections of the filaments in the network structure-cell composite touch one another at least partially, and fixing the folded network structure-cell composite with subsequent dissolution of the network structure are provided.

By coating the network structure with the protein material and populating the coated network structure before the network structure is dissolved from the carrier substrate, the carrier substrate advantageously performs its support function until the cells are arranged in the composite, wherein the arrangement of the filaments and thus of the blood vessels formed subsequently is retained. By folding the network structure-cell composite to form a multilayer, the formation of a spatial form of the tissue product is advantageously facilitated.

Advantageously, different variants of the folding of the network structure-cell composite are available, which can be carried out individually or in combination. According to a first variant, it is provided that the network structure-cell composite is hung by way of an elongate holding element, such as, for example, a holding thread or a holding rod, in such a manner that surfaces of the network structure-cell composite at which the lateral sections of the filaments are exposed touch one another. The network structure-cell composite is folded over at the elongate holding element so that two sections of a surface of the network structure-cell composite that are separated by the elongate holding element lie against one another. The sections of the surface advantageously adhere to one another, so that a closed cell composite is formed, from which the elongate holding element can easily be separated.

According to a second variant (“origami” variant), it is provided that the network structure-cell composite is placed on a folding substrate in such a manner that the lateral sections of the filaments are exposed, and the folding substrate is deformed in such a manner that surfaces of the network structure-cell composite at which the lateral sections of the filaments are exposed touch one another. The deformation of the folding substrate advantageously provides a force effect under which the sections of a lateral surface of the network structure-cell composite touch one another, whereby the adherent connection of the parts of the network structure-cell composite is assisted.

According to a third variant, it is provided that the network structure-cell composite is placed on a folding tool in such a manner that the lateral sections of the filaments are exposed, and the folding tool is operated in such a manner that surfaces of the network structure-cell composite at which the lateral sections of the filaments are exposed touch one another. In this case too, a force is advantageously exerted by the folding tool, under which force the cell composite is stabilized in the folded state.

Particularly preferably, the network structure is formed mirror-symmetrically with respect to a predetermined reference plane perpendicular to the extent of the network structure, wherein folding of the network structure-cell composite takes place along the reference line. Accordingly, filament sections of identical forms touch one another after folding, so that larger vessel diameters are achieved after the dissolution.

According to an alternative embodiment of the invention, it is provided that the network structure is coated with the protein material and the coated network structure is populated with the endothelial cells and with the tissue-forming biological cells after the network structure has been dissolved from the carrier substrate. In this case, advantages can be achieved in that the supply of the protein material, of the endothelial cells and of the tissue-forming biological cells into the network structure from multiple sides is facilitated.

According to a further preferred embodiment of the invention, the production of the network structure preferably comprises 3D deposition of the filaments without binding to a solid carrier substrate. The 3D deposition can be carried out, for example, in a high-viscosity carrier liquid or a soft polymer, in particular a substrate block, or on a layer of prefabricated filaments.

The term “3D deposition” refers generally to methods with which a three-dimensional structure is produced from the filaments by building up the support polymer with or without substrate binding. The use of 3D deposition has the particular advantage that the distribution of the filaments and thus of the blood vessels in the vascularized tissue can be defined by the spatially resolved control of the deposition of the support polymer.

The 3D deposition of the filaments may advantageously comprise 3D freeze printing of the support polymer, in which a solution of the support polymer is built up layer by layer at a temperature below the freezing point of the support polymer and crosslinked, so that the arrangement of the filaments with an inner volume is formed.

Alternatively, the 3D deposition of the filaments may comprise extrusion of the support polymer into a carrier material by means of a cannula device. Particularly preferably, the cannula device comprises a coaxial cannula with which the support polymer and the endothelial cells may be introduced into the carrier material at the same time.

The support polymer preferably comprises alginate, wherein the support polymer is dissolved using alginate lyase. The use of alginate has particular advantages owing to the biocompatibility of alginate and its suitability for residue-free dissolution to form the vessels in the cell composite. Alternatively or in addition, other uronic acid-based polysaccharides, such as, for example, galacturonic acid, or protein-based support polymers, such as, for example, gelatin, can be used. The support polymer is preferably dissolved using alginate lyase, dextranase and/or pectinase. Furthermore, a complexing agent, such as, for example, EDTA (ethylenediaminetetraacetic acid), may be used for dissolving the support structure.

A particular advantage of the invention consists in that the vascularized tissue can be produced in freely selectable sizes. It can advantageously be provided that at least two layers of vascularized tissue are connected to form a tissue block.

The method according to the invention advantageously allows a modification of the cell composite for forming the vascularized tissue, for example in order to improve its supply.

According to an embodiment of the invention, an embedding of least one perfusion line into the cell composite for producing the vascularized tissue, into the vascularized tissue and/or into the tissue block is preferably provided, wherein the perfusion line is produced from a soluble material and is arranged to supply a culture medium. The perfusion line allows, for example in a first culture phase of the vascularized tissue, an increased supply of culture medium in order to assist with cell propagation in the vascularized tissue, wherein the perfusion line can be dissolved in a later culture phase of the vascularized tissue or during use thereof, when the inner vessels are sufficiently thick.

Further details and advantages of the invention will be described in the following text with reference to the accompanying drawings. The drawings show schematically in:

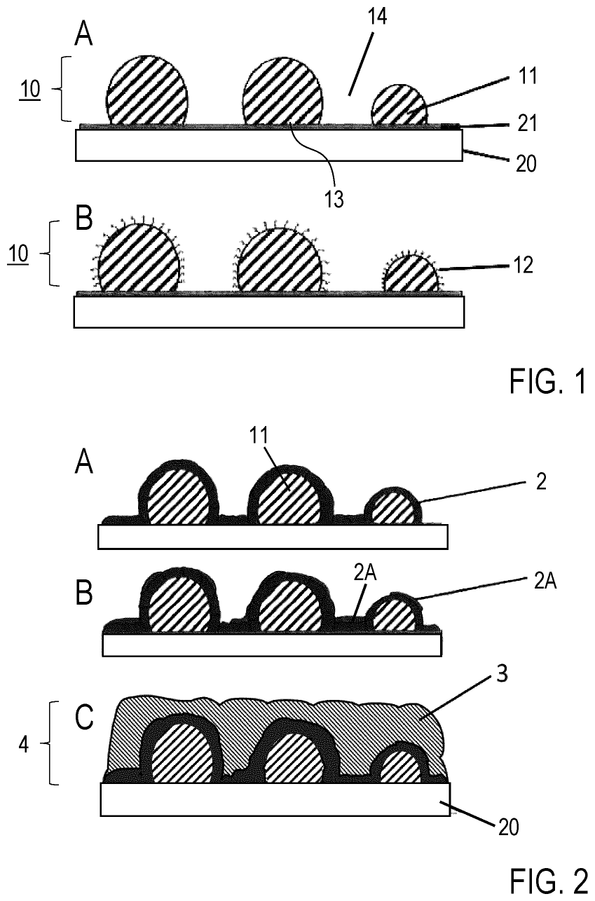

FIGS. 1 to 5: features of the method for producing vascularized tissue according to an embodiment of the invention,

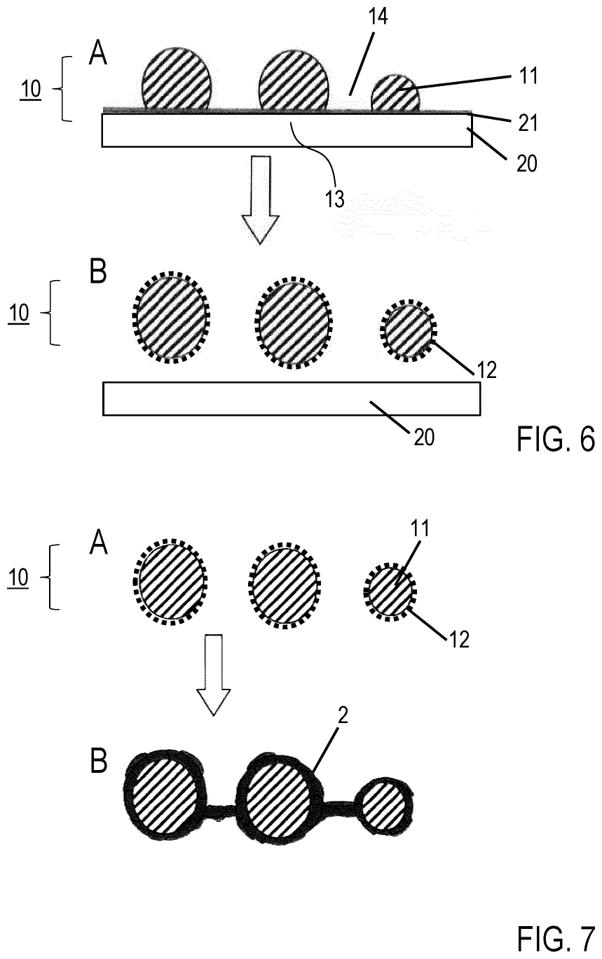

FIGS. 6 to 9: features of the method for producing vascularized tissue according to further embodiments of the invention,

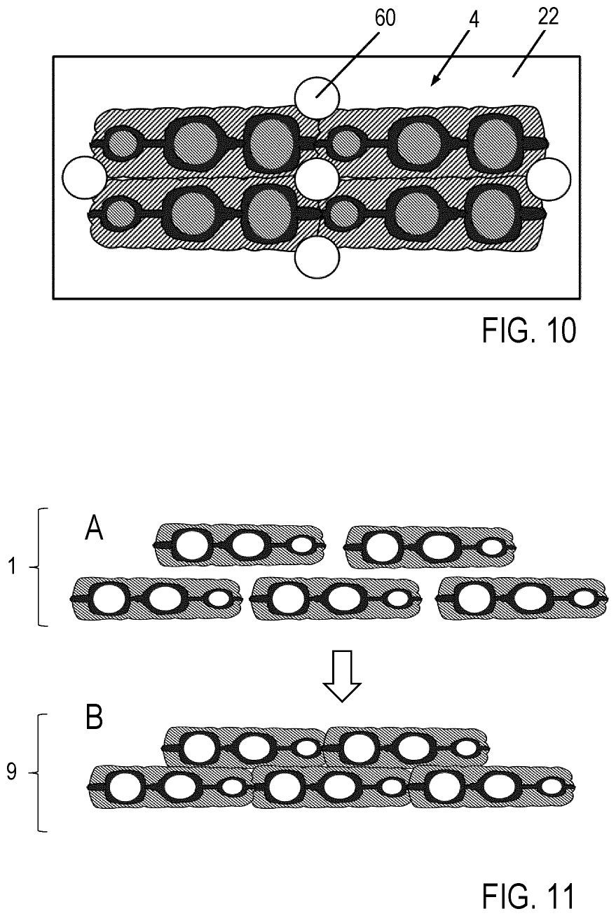

FIG. 10: the production of vascularized tissue with embedded perfusion lines, and

FIG. 11: the production of vascularized tissue in the form of a tissue block.

Features of embodiments of the invention will be described in the following text with reference, by way of example, to the production of vascularized cardiac muscle cell tissue. It is emphasized that the implementation of the invention in practice is not limited to the application with cardiac muscle cells but is also possible with other cell types, such as, for example, liver cells, kidney cells, nerve cells and/or pancreatic cells. Details of the specific cell types of endothelial cells and cardiac muscle cells that are used, the preparation, for example by differentiation from pluripotent stem cells, and culturing thereof will not be described because they are known per se from the prior art.

The figures illustrate embodiments of the invention by means of enlarged sectional views which each show a single-layer or two-layer detail of a network structure or of a vascularized tissue in the region of a small number of filaments or a small number of vessels. In practice, the invention is implemented with extensive network structures which can comprise considerably more filaments or vessels and/or more layers or a spatial form of the network structure.

Vascularized biological tissue is produced using techniques which are known per se from biotechnological tasks in the laboratory or from industrial production, such as, for example, using incubators having devices for supplying solutions and/or suspensions.

FIGS. 1 to 5 illustrate a first embodiment of the method according to the invention for producing vascularized tissue, in which the production of a network structure and the population thereof with endothelial cells and cardiac muscle cells is carried out on a solid carrier substrate.

FIGS. 1A and 1B show the production of the network structure 10 from a plurality of interconnected filaments 11 of a support polymer, such as, for example, alginate, and the coating of the network structure 10 with a protein material 12, such as, for example, fibronectin or laminin.

The solid carrier substrate 20 having preferably a planar surface is produced, for example, from PMMA or glass. The carrier substrate 20 is formed, for example, by the base of a well of a microtiter plate (microwell) and coated with a matrix material 21, such as, for example, dextran. The thickness of the layer, which is preferably closed, of matrix material 21 is, for example, 10 μm.

The filaments 11, which are shown in a sectional view in the figures and can touch one another and/or intersect outside the plane of the drawing, are arranged according to FIG. 1A, for example, in the form of a prefabricated network of precipitated alginate filaments or by 3D deposition on the carrier substrate 10 in a disordered distribution or with a regular distribution. Lateral sections 13 of the filaments 11 touch the coated carrier substrate 10, and gaps 14 remain between the filaments 11. The diameters of the alginate filaments 11 are preferably chosen in the range of from 50 μm to 500 μm or even above 500 μm. The gaps 14 have a size in the range of from 100 μm to 500 μm.

According to FIG. 1B the protein material 12 is deposited, for example, from a surrounding solution over the coated carrier substrate 20 or by purposive drop deposition on the filaments 11 and the exposed regions of the matrix material 21.

Then, according to FIG. 2, formation of a network structure-cell composite 4 is carried out. To this end, the filaments 11 and the exposed regions of the matrix material 21 are first populated in an adherently adhering manner with a first type of endothelial cells 2 (see FIG. 2A). The cells are supplied from a suspension, which covers the coated carrier substrate 20 and the filaments 11. A closed layer (monolayer or multilayer) of the endothelial cells 2 is preferably formed. Optionally, the filaments 11 and/or the regions between the filaments 11 are additionally populated with a second type of cells 2A, such as, for example, smooth muscle cells (see FIG. 2B). By using two or more types of endothelial cells, the composition of vessel walls can advantageously be formed similarly to that in natural tissues. Optionally, a cultivation of the endothelial cells 2, 2A in the adherent state on the carrier substrate 20 with the supply of a culture medium can be provided, wherein the endothelial cells 2, 2A propagate.

The tissue-forming cells 3, such as, for example, cardiac muscle cells, are then disposed on the endothelial cells 2, 2A (see FIG. 2C). The cardiac muscle cells are again supplied from a suspension which covers the coated carrier substrate 20 and the filaments 11 provided with the endothelial cells 2, 2A. The tissue-forming cells 3 form a closed layer which extends over the filaments 11 and the regions between the filaments 11. The overall thickness of the network structure-cell composite 4 so produced is, for example, 5 mm.

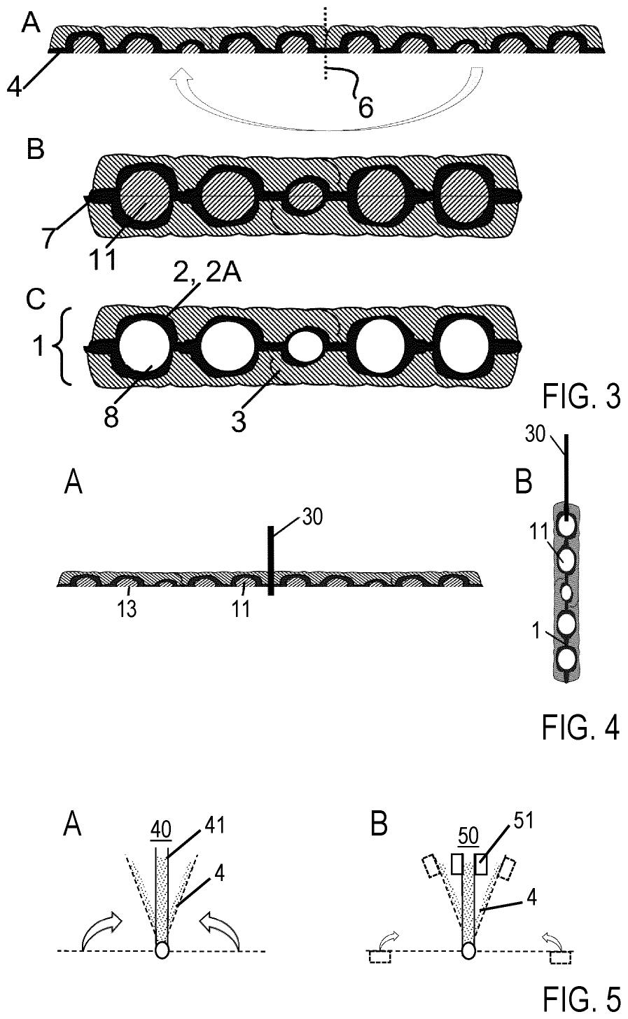

The network structure-cell composite 4 is dissolved from the carrier substrate 20. To this end, the matrix material 21 is dissolved by the supply of a solvent which dissolves the matrix material 21 but not the support polymer of the filaments, such as, for example, dextranase, so that the network structure-cell composite 4 separates from the carrier substrate 20 (see FIG. 3A).

The network structure 10 is preferably formed mirror-symmetrically with respect to a predetermined reference plane 6 perpendicular to the extent of the network structure 10, as is shown schematically in FIG. 3A. By folding the network structure-cell composite 4 at the reference plane (see arrow in FIG. 3A), the filaments that are in the same positions relative to the fold line 7 and are of the same size match up (see FIG. 3B). From the filaments 11, which were initially oblate at the side as a result of contact with the carrier substrate 20, there are formed filaments which are rounded on all sides and the cross-sectional form of which is adapted to the cross-sectional form of the vessels to be formed. In the folded state, surfaces of the network structure-cell composite 4 that touch one another are connected together, whereby the network structure-cell composite 4 is fixed.

Symmetry with respect to the reference plane 6 is not a necessary feature of the invention. Even in the case of non-symmetrical distributions, folding of the network structure-cell composite 4, as a result of self-organization processes, yields a distribution of touching filaments 11, which are provided for the subsequent vascularization.

Subsequently, dissolution of the network structure 10 is carried out, so that the vascularized tissue 1 is formed (see FIG. 3C). Dissolution of the network structure 10 is carried out, for example, by supplying alginate lyase. As a result, the vascularized tissue 1 is composed of the tissue-forming cells 3, in the composite of which hollow spaces remain after dissolution of the filaments. The hollow spaces are connected together, so that they form vessels 8 in the vascularized tissue 1. Inside walls of the vessels 8 are formed by the endothelial cells 2, 2A.

FIGS. 4 and 5 show variants of the folding of the network structure-cell composite 4 by hanging it from an elongate holding element 30 (FIG. 4), using a folding substrate 40 (FIG. 5A) or using a folding tool 50 (FIG. 5).

The elongate holding element 30 is, for example, a holding thread over which the network structure-cell composite 4 is placed along the line of intersection of the reference plane 6 with the network structure-cell composite 4 (see FIG. 4A). Surfaces of the network structure-cell composite 4 at which the lateral sections 13 of the filaments 11 are exposed swing on both sides of the holding element 30 under the action of gravity until they touch and connect to one another by the formation of cell-cell contacts (see FIG. 4B). The holding element 30 can then be removed and the filaments 11 dissolved to form the vascularized tissue 1.

The folding substrate 40 is a foldable carrier element having two planar wings, which is pivotable between an unfolded, planar state (shown by broken lines FIG. 5A) and a folded-together state (shown by solid lines in FIG. 5A). The folding substrate 40 consists, for example, of an elastically resilient, bistable material, which is pivotable between the two states by internal mechanical stresses. By means of the folding substrate 40, two parts of a network structure-cell composite 4 can be placed against one another in a planar manner and contacted. The network structure-cell composite 4 is placed on the folding substrate 40 in the unfolded state, wherein the line of intersection of the reference plane 6 with the network structure-cell composite 4 (see FIG. 3) coincides with the run of the pivot axis between the two wings of the folding substrate 40. As a result of being transferred into the folded-together state (see arrows), the folding substrate 40 is deformed in such a manner that surfaces of the network structure-cell composite 4 at which the lateral sections of the filaments are exposed touch one another and are connected together by the formation of cell-cell contacts. The folding substrate 40 can then be removed and the filaments dissolved to form the vascularized tissue.

The folding tool 50 according to FIG. 5B has a similar function to the folding substrate 40 in that it is pivotable between an unfolded, planar state (shown by broken lines in FIG. 5B) and a folded-together state (shown by solid lines in FIG. 5B). Unlike the internal stress of the folding substrate 40, the folding tool 50 is operated by at least one actuator 51 in order to fold the network structure-cell composite 4.

FIGS. 6 to 8 illustrate a second embodiment of the method according to the invention for producing vascularized tissue, in which only the production of the network structure takes place on a solid carrier substrate. The network structure is then dissolved from the carrier substrate, coated with a protein material and populated with endothelial cells and cardiac muscle cells.

FIG. 6A shows the production of the network structure 10 from a plurality of interconnected filaments 11 of a support polymer, such as, for example, alginate, with mutual distances 14 on the carrier substrate 20 coated with a matrix material 21, as in FIG. 1A. The network structure 10 is then dissolved from the carrier substrate 20 and coated on all sides with the protein material 12 (FIGS. 6B and 7A).

The network structure 10 dissolved from the carrier substrate 20 and coated with the protein material 12 is populated with endothelial cells 2 (FIG. 7B). To this end, the network structure 10 is incubated in an incubator with a suspension of the endothelial cells 2.

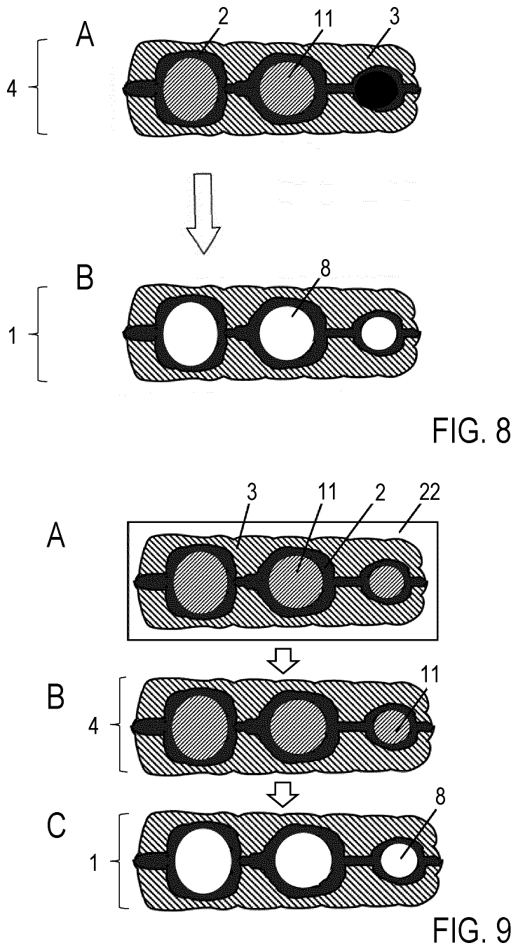

By incubation with a cardiac muscle cell suspension, the endothelial cells 2, which are arranged on and between the filaments 11, are populated with cardiac muscle cells 3, whereby the network structure-cell composite 4 is formed (FIG. 8A). The support polymer of the filaments 11 is then dissolved, whereby inside the network structure-cell composite 4 and thus the vascularized tissue 1 is formed.

FIG. 9 illustrates a third embodiment of the method according to the invention for producing vascularized tissue, in which the production of the network structure and population with endothelial cells and cardiac muscle cells are carried out not with binding to a solid substrate, but in a substrate block, for example of a soft, incompletely crosslinked alginate. The alginate is formed to be sufficiently soft that cells are able to align in the alginate and at the same time are separated from their surroundings. The network structure is then dissolved from the substrate block and the support polymer of the filaments is dissolved.

According to FIG. 9A, the filaments 11 of the support polymer, such as, for example, alginate or galacturonic acid, are embedded by means of a cannula. Preference is given to the use of a coaxial cannula, with the inner channel of which there is supplied the support polymer, in particular weakly crosslinked alginate and optionally extracellular matrix (ECM) for polarization of the cells, and with the outer channel of which there are supplied the endothelial cells 2 together with ECM and optionally also an addition of ethylenediaminetetraacetic acid (EDTA). The coaxial cannula is placed in the substrate block 22 in accordance with a predefined program and moved, for example withdrawn along predefined paths, while delivering the support polymer and the endothelial cells 2 with the additives. Cardiac muscle cells 3 with additives of ECM and optionally also EDTA are then supplied and placed on the endothelial cells 2 by means of a cannula, so that the network structure-cell composite 4 is formed in the substrate block 22.

In the further method, the network structure-cell composite 4 is separated from the substrate block 22 in that the substrate block 22 is dissolved with alginate lyase (FIG. 9B). The filaments 11 are then dissolved, for example with pectinase, whereby the free vessels 8 of the vascularized tissue 1 are formed (FIG. 9C).

FIG. 10 illustrates a modification of the method according to FIG. 9, wherein there are additionally embedded in the network structure-cell composite 4 perfusion lines 60 for additionally supplying the cells with culture medium, in particular while the network structure-cell composite 4 is being cultured in the substrate block 22. The perfusion lines 60 serve in particular for supply during the maturation and organization of the network structure-cell composite 4, and they are preferably produced from a degradable material, such as, for example, dextran. For example, dialysis tubes can be used as perfusion lines 60. When the network structure-cell composite 4 is separated from the substrate block 22, the perfusion lines 60 can be dissolved, for example with dextranase.

Multiple sections of the vascularized tissue 1 which has been produced in particular by a method according to one of the described embodiments can be connected to form a tissue block 9, as is shown schematically in FIG. 11. Connection takes place by mutual contacting with the addition of genipin.

The features of the invention which have been disclosed in the preceding description, the drawings and the claims can be of importance both individually and in combination or sub-combination for implementing the invention in its various embodiments.

1. A method for producing vascularized biological tissue, comprising the steps of
producing a network structure from a plurality of interconnected filaments of a support polymer,
coating the network structure with a protein material,
populating the coated network structure with endothelial cells and with tissue-forming biological cells, and
dissolving the filaments of the network structure so that the vascularized tissue is formed.

2. The method according to claim 1, wherein
the producing of the network structure comprises depositing the filaments on a carrier substrate coated with a degradable matrix material and subsequently dissolving the network structure from the carrier substrate.

3. The method according to claim 2, further comprising the steps of
forming a network structure-cell composite by coating the network structure with the protein material and populating the coated network structure with the endothelial cells and with the tissue-forming biological cells, before the network structure is dissolved from the carrier substrate, wherein lateral sections of the filaments in the network structure-cell composite touch the carrier substrate,
dissolving the network structure-cell composite from the carrier substrate,
folding the network structure-cell composite to form a multilayer, in such a manner that the lateral sections of the filaments in the network structure-cell composite touch one another at least partially, and
fixing the folded network structure-cell composite with subsequent dissolution of the network structure.

4. The method according to claim 3, wherein the folding of the network structure-cell composite comprises
hanging the network structure-cell composite over an elongate holding element, so that surfaces of the network structure-cell composite at which the lateral sections of the filaments are exposed touch one another.

5. The method according to claim 3, in wherein
the network structure is formed mirror-symmetrically with respect to a predetermined reference plane perpendicular to an extent of the network structure, and
folding of the network structure-cell composite takes place along the reference plane.

6. The method according to claim 2, wherein
the network structure is coated with the protein material and the coated network structure is populated with the endothelial cells and with the tissue-forming biological cells after the network structure has been dissolved from the carrier substrate.

7. The method according to claim 1, wherein
the production of the network structure comprises 3D deposition of the filaments without binding to a solid carrier substrate.

8. The method according to claim 7, wherein
the 3D deposition of the filaments comprises 3D freeze printing of the support polymer.

9. The method according to claim 7, wherein
the 3D deposition of the filaments comprises extrusion of the support polymer into a carrier material using a cannula device.

10. The method according to claim 9, wherein
the cannula device comprises a coaxial cannula with which the support polymer and the endothelial cells are introduced into the carrier material at the same time.

11. The method according to claim 1, having at least one of the following features
the support polymer comprises at least one of alginate, another uronic acid-based polysaccharide; and a protein-based support polymer, and
the support polymer is dissolved using at least one of alginate lyase, dextranase, pectinase and a complexing agent.

12. The method according to claim 1, further comprising
connecting at least two layers of vascularized tissue to form a tissue block.

13. The method according to claim 12, further comprising
embedding at least one perfusion line into the tissue block, wherein the perfusion line is produced from a soluble material and is arranged to supply a culture medium into the tissue block.

14. The method according to claim 1, wherein
the tissue-forming biological cells comprise at least one of cardiac muscle cells, liver cells, kidney cells, nerve cells and pancreatic cells.

15. A method according to claim 3, wherein the folding of the network structure-cell composite comprises
placing the network structure-cell composite on a folding substrate in such a manner that the lateral sections of the filaments are exposed, and deforming the folding substrate in such a manner that surfaces of the network structure-cell composite at which the lateral sections of the filaments are exposed touch one another.

16. A method according to claim 3, wherein the folding of the network structure-cell composite comprises
placing the network structure-cell composite on a folding tool in such a manner that the lateral sections of the filaments are exposed, and operating the folding tool in such a manner that surfaces of the network structure-cell composite at which the lateral sections of the filaments are exposed touch one another.

17. The method according to claim 1, further comprising
embedding at least one perfusion line into the vascularized tissue, wherein the perfusion line is produced from a soluble material and is arranged to supply a culture medium into the vascularized tissue.
